(19) United States
(12) Patent Application Publication
(10) Pub. No.: US 2009/0036956 A1
(43) Pub. Date: Feb. 5, 2009

(54) LASER TREATMENT SYSTEM AND RELATED METHODS

(76) Inventors: Victor Alexeevich Mikhalylov, Moscow (RU); Alexander Iosifovich Zagumennyy, Moscow (RU); Petr Andreevich Gonchar, Moscow (RU); Yury Dmitrievich Zavartsev, Moscow (RU); Yury Lvovich Kalachev, Moscow (RU); Vladimir Vladimirovich Podreshetnikov, Moscow (RU); Anatoly Andreevich Sirotkin, Moscow (RU); Sergey Alexandrovich Koutovoi, Krasnodar (RU)

Correspondence Address: THOMAS LOOP, P.O. BOX 21466, SEATTLE, WA, 98111, US

(21) Appl. No.: 12/113,154

(22) Filed: Apr. 30, 2008

Related U.S. Application Data
(60) Provisional application No. 60/915,087, filed on Apr. 30, 2007.

(51) Int. Cl. A61N 5/067; H01S 5/024

(52) U.S. Cl. 607/89; 372/34

(57) ABSTRACT

The invention disclosed herein related to laser systems, arrangements, and related methods for irradiating a selected biotissue with continuous or pulsed laser radiation having wavelengths selected from the group of 0.6 to 1.06 microns, 1.34 to 1.70 microns, 1.8 to 2.2 microns, and 2.4 to 3.1 microns. The laser radiation wavelengths may be varied continuously or discretely.

### CROSS-REFERENCE TO RELATED APPLICATION

This application claims the benefit of U.S. Provisional Application No. 60/915,087 filed on Apr. 30, 2007, which application is incorporated herein by reference in its entireties for all purposes.

### TECHNICAL FIELD

This invention relates to medical solid-state lasers and has a wide range of applications within the medical industry, as well as some cosmetological fields. The most promising use of this invention is within opthalmology for the treatment of dartous kertitis, laser compensation of a hyperopia and an astigmatism, treatment of a cataract, a glaucoma, for laser hardening of the capillaries, for a laser thermocoagulation, a hyperthermia, kerf and evaporation of tissues, and in otorhinolarygology and rhino surgery.

### BACKGROUND OF THE INVENTION

The method of biotissue treatment using laser radiation is well known to those in the art. In 1999 a presentation was given at the 97^{th} DOG Annual Meeting on this subject (“Diode laser thermokeratoplasty for hyperopia correction-results of a two-center study,” M. Derse et al., German translation.) The method used in this study used radiation generated from a semiconductor laser with a wavelength of 1.9 microns to attack targeted biotissues within the eye. The apparatus used to generate the non-pulsed radiation comprised a laser head with a power package, the block of delivery and focusing of radiation.

The study performed by M. Derse et al. and presented at the 97^{th} DOG Annual Meeting did present some limitations, however. The apparatus, as it was designed, was only capable of producing a fixed wavelength of radiation, in this case, 1.9 microns. Consequently, this limited the depth of coagulation of biotissues the radiation was able to treat. Most medical procedures, such as the treatment of a cornea, require the device to have the capability of modifying the depth of radiation due to the biotissues' three-dimensional nature. Furthermore, the lack of control over radiation variance also gives rise to the danger of transit of the laser through an appreciable part of healthy biotissues in order to access a deeper laying biotissues needing treatment. Furthermore, there is no variance control to allow for the various absorption rates of radiation for various depths of coagulated biotissues. This particular limitation does not allow the process to be optimized during thermokeratoplatic and subsequently cannot achieve the most stable and exact compensation of vision and an astigmatism. The fixed wavelength further limits the treatment. Given these restrictions of the system, the laser produces a rather in-homogeneity of a thermocoagulation in the volume of coagulated biotissues with the maximal heating an entrance surface biotissues is characteristic.

A method of utilizing a radiation of Ho:YAG laser with lamp pump to effect the biotissues of an eye for the purpose of reversal of hyperopia after myopic photorefractive keratectomy was introduced in 1997. (“Holmium laser thermokeratoplasty for the reversal of hyperopia after myopic photorefractive keratectomy,” British Journal of Optometry, 1997, v. 81, pp 541-543.) This method for laser opthalmology thermocoagulator for thermokeratoplasty comprises a laser head with ha pulse Ho:YAG laser with lamp pump; a block of delivery; and a fiber optic cable with an optical system allocated on a yield of a fiber optical contact or contactless cord terminal for the purpose of focusing radiation. Again, however, the method is limited by the laser radiation only on one of the fixed lengths of waves in the field of 2.1 micros, which provides only fixed heat penetration of biotissues, that in most cases is not optimum from the point of view of affecting a laser radiation on biotissues of an eye wherein there are different depths and transparencies for various patients. It, for example, in thermokeratoplasty, results in the reduction of accuracy, reproducibility of compensation of vision and an astigmatism and by that to deterioration of accuracy of compensation of vision. The fixed wavelength of radiation of the Ho:YAG laser does not allow to alter the depth of coagulation of biotissues heated by radiation. Currently, there are alternate technologies, which are less expensive and have a longer useful life with less demanding periodic service. All of these technologies, however, still produce inhomogeneity of a therocoagulation in volume of coagulated biotissues.

### SUMMARY OF THE INVENTION

To overcome the prior art's technical problem of dilation of functionalities, the current invention carries out various types of operations with the use of coagulation, ablation, a carbonization, cauterizing or kerf of biotissues, controlling depth of coagulation of biotissues by varying the wavelength emitted from the laser, and augmentation of the uniformity of thermocoagulation of biotissues in a direction affecting laser radiation. An addition difficulty that also is resolved is the combined effect on the biotissues exposed to both laser radiation and a direct thermal action of the warmed end of an optical system wherein only the biotissues exposed to the laser radiation are to be therapeutically treated.

This invention allows for the remedy of one problem, in part, in one of the bands of wavelengths between 0.96-1.06 microns, 1.34-1.70 microns, 1.8-2.2 microns, and 2.3-3.1 microns by varying the wavelength of radiation and applying the radiation continuously or discretely. The invention also allows for the attack of biotissues by an additional laser radiation, at least, in one of the bands of wavelengths from the group 0.47-0.49 microns, 0.62-0.65 microns, 0.8-0.95 microns and 1.25-1.29 microns.

These known bands of radiation wavelengths are utilized within the invention and are used to accommodate the execution of radiation of various wavelengths, while incorporating factors such as absorption. The apparatus itself comprises a laser head with the power package, the block of delivery and focusing device of the radiation, a fiber-optic cable on which the cable is mounted to the laser head, a laser head executed with an opportunity of radiation on various lengths of waves with the limits of, at least, one absorption band of biotissues, and the block of delivery and focusing of radiation follow-up further comprising a transparent heat conducting sheet, the block of refrigerating sheet and the block of an exposure of refrigeration.

The laser head itself, must be executable with a wavelength of radiation within the limits of, at least, one absorption band of biotissues. Furthermore, the laser head can contain not less than two lasers emitting various wavelengths. The emitting devise may be comprised of a diode laser, a fiber laser or a solid-state laser, which hose can be executed from a single crystal from the following group: YAlO₃, Y₃Al₅O₁₂, YVO₄, GdVO₄, YLiF₄. Thus the optically active element of the laser can be allowed, at least, by one ion from group Nd³⁺, Pr³⁺, Ho³⁺, Er³⁺, Tm³⁺, Yd³⁺, Ti³⁺, Cr³⁺, Cr⁴⁺.

The laser head can contain a block lamp or a diode pumped component. This arrangement allows data of radiation of various lengths of waves in one beam, in addition to further laser radiation executed from a semiconductor laser.

In particular, the focusing block and deliveries of radiation electrically can be connected to the power package.

In particular, the cord terminal can contain the case and can be mounted in such a manner that the optical fibre jut out of the case. Thus the optical fibre can jut out of the case on the controlled distance. Thus face and lateral cylindrical surfaces of the optical fibre jutting out of the case, can absorb not less than 10% of power of forwarded radiation.

In particular, the transparent heat-conducting sheet can be mounted behind a the cord terminal and is connected to the block of refrigerating of a sheet.

In particular, the block of refrigerating electrically can be connected to the block of an exposure of refrigerating.

In particular, the arrangement can follow-up contain a thermal gauge mounted on a heat-conducting sheet.

The declared inventions representing a method and the arrangement for its implementation, are connected by a uniform inventor's plan.

### DETAILED DESCRIPTION OF THE DRAWINGS

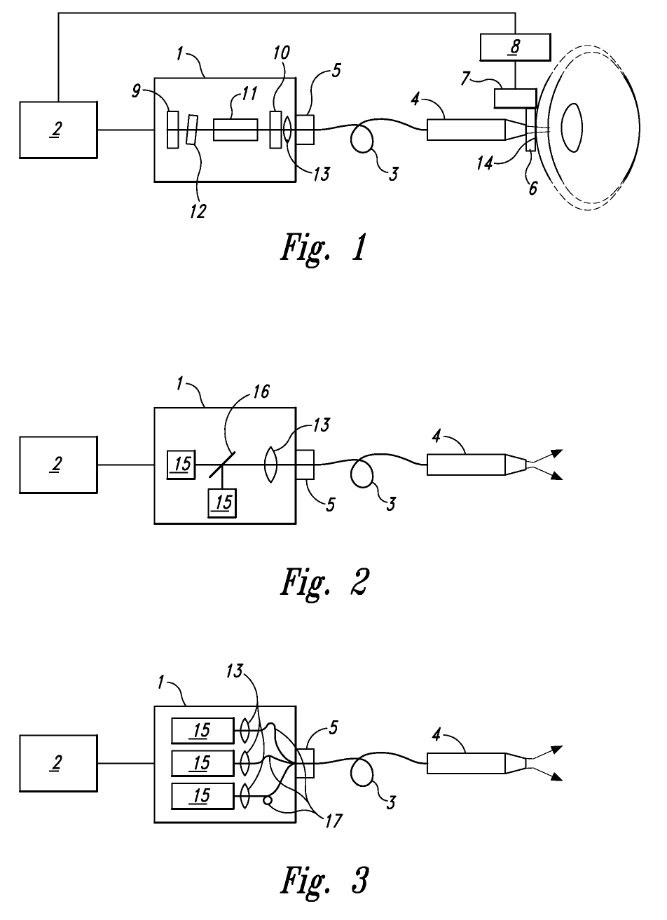

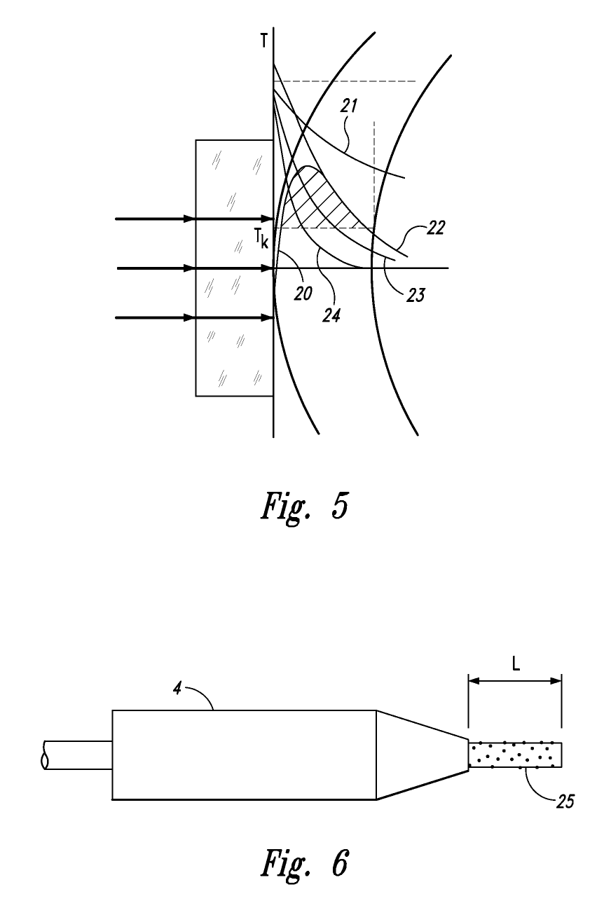

Declared inventions are illustrated by the drafts where on FIG. 1-FIG. 4 instances of concrete accomplishment laser ophthalmologic thermocoagulation are shown, on FIG. 5 the examples of temperature T in cross-section of a cornea of an eye are presented, and on FIG. 6 the instance of concrete accomplishment of the cord terminal is shown.

In case of use of the tunable laser the arrangement (FIG. 1) contains a laser head 1, the power package 2, the block of delivery and focusing of radiation into which components are included the fibre-optic cable 3, the focusing block of radiation 4 and a fibre-optic demountable connector 5, a transparent heat-conducting sheet 6, the block of refrigerating of a sheet 7 and the block of an exposure of refrigerating 8. The laser head 1 contains completely reflecting 9 and target 10 mirrors of the resonator of a laser head, the optically active element 11, dispersing element 12, executed, for example, in the form of a prism, filter Lio, and etc., and system of optical connection of the radiation 13, providing input of radiation in face of an optical fibre of the cable 3 placed inside of a fibre-optic demountable connector 5. The optically active element 11 has wide continuous or a line radiation spectrum in the field of absorption bands of a biotissue which practically coincides with absorption bands of water. Rearrangement of a wavelength of radiation of a laser head 1 is carried out by means of a dispersing element 12. On a surface of the sheet 6 which are being in contact to biotissues, the torque motor of contact 14 is had.

In case of use of two diode lasers 15, in particular, with various lengths of waves within the limits of width of absorption bands of a biotissue the arrangement (FIG. 2) follow-up contains a polarizer 16, providing overlapping of beams of radiation of lasers 15 in space.

In case of use of three diode lasers 15 with various lengths of waves within the limits of width of absorption bands of biotissues in the arrangement (FIG. 3) beams of radiations from each laser 15 are focused in faces of separate optical fibres 17, and summation of beams of radiation is carried out in a demountable connector 5.

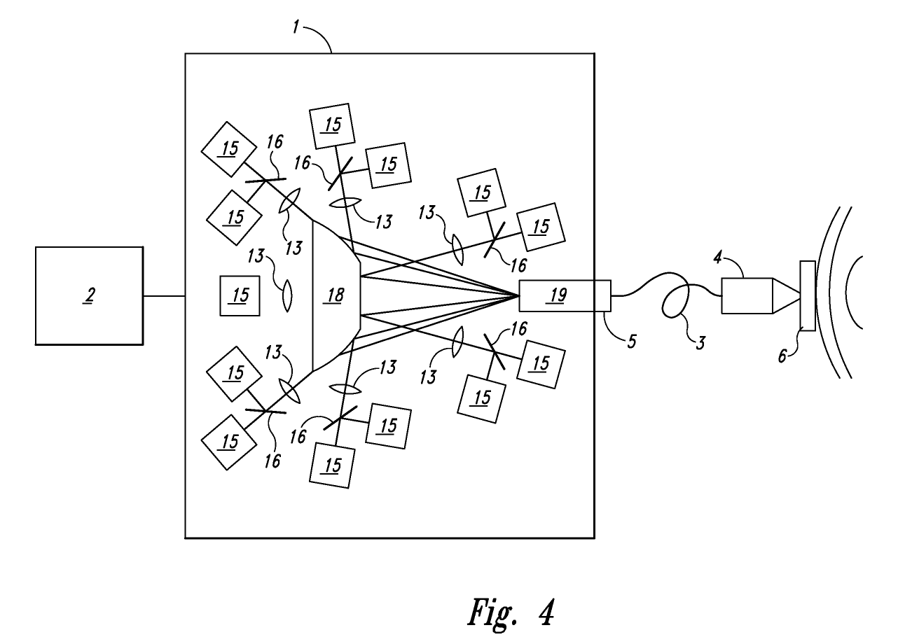

On FIG. 4 the instance of concrete accomplishment with use of the thirteen diode lasers 15) is presented, and lasers 15 can have various lengths of waves of radiation, including, and in visible range of a spectrum within the limits of range of lengths of waves 0.47-0.49 microns, 0.62-0.65 microns, 0.8-0.95 microns, 1.25-1.29 microns (ranges of therapeutic act). Radiation of lasers 15 is in pairs matched in several beams, which after reflectance from a many-sided prism 18 are referred to system of data 19 where all beams are input into a fibre-optical cable 3. Depending on a solved problem the quantity of diode lasers 15 can be enlarged, for example, by increase of quantity of reflecting sides of a prism 18.

As an active element of the tunable laser (FIG. 1) for absorption band of a biotissue 1.34-1.70 microns it is possible to use the Nd-containing crystal YAlO₃ emitting on lengths of waves of 1.3414 microns, 1.3777 microns, 1.3842 microns, 1.4020 microns and 1.4325 microns, the Nd-containing crystal Y₃Al₅O₁₂ emitting on lengths of waves of 1.3381 microns, 1.3572 microns, 1.4140 microns and 1.4444 microns which are Nd-containing crystals YVO₄ and GdVO₄, emitting on lengths of waves 1.34-1.38 microns.

As the active elements 11 (FIG. 1) with rearrangement of a wavelength within the limits of absorption bands of biotissues 0.96-1.06 microns, 1.34-1.70 microns, 1.8-2.2 microns, 2.4-3.1 microns can be used as well other crystals containing ions of neodymium Nd³⁺, praseodymium Pr³⁺, holmium Ho³⁺, erbium Er³⁺, thulium Tm³⁺, ytterbium Yb³⁺, chromes Cr³⁺ and Cr⁴⁺, titanium Ti³⁺, and also the glasses containing specified ions. Besides it is possible to use parametric transformation, and also a frequency doubling of radiation of these lasers.

For continuous rearrangement of a wavelength of radiation in the field of 2.8-3.1 microns it is possible to use the laser with an active component 11 (FIG. 1) from the crystal Y₃Sc₂Ga₅O₁₂ containing ions Cr³⁺, Yb³⁺ and Ho³⁺ or ions Cr³⁺ and Er³ from the crystals YAlO₃, Y₃Al₅O₁₂ containing ions Cr³⁺, and also others erbium and holmium solid-state crystalline lasers.

For range 1.34-1.44 microns it is possible to use low-cost diode lasers 15 (FIG. 2). Rearrangement of a wavelength of radiation of diode lasers in small limits is carried out by change of temperature of an active component. Rearrangement of a wavelength of radiation in wider limits (over a complete bandwidth of absorption line) is provided with several diode lasers 15 (FIG. 2-FIG. 4) with various emitting wavelengths and beam convergence, in particular, by means of system of data 19 (FIG. 4), operating in turn.

For range of absorption band 1.8-2.2 microns it is possible to use a set of diode lasers 15 with various wavelengths of emitting within the limits of complete width of this absorption band. Thus the penetration depth of radiation and, accordingly, depth of coagulation of tissues can vary over a wide range (0.05-1 mm) unlike a case of use only one diode laser with the fixed wavelength of radiation 1.9 microns that allows to change the depth of a radiation effect to biotissues at carrying out of various types of operations with use of coagulation, ablation, a carbonization and cutting of tissues.

The arrangement (FIG. 1) works as follows. Output radiation of a laser head 1 by means of system of optical linking of radiation 13 is focused in face of a fibre 3 which forwards radiation up to a the cord terminal 4. On an end of a cord terminal 4 radiation is focused so that focus was on some distance from its output end. The position of focus is fixed by means of the mechanical catch having on the output end of the cord terminal 4 an opportunity of adjustment of distance between the cord terminal 4 and biotissue exposing to a laser radiation. The distance between a biotissue and a place exposed to a radiation is possible to define visually by a radiation focusing spot of the semiconductor laser of the visible band which has been preliminarily built in an emitter 1.

One of optional versions is the use of a slit lamp. In this case the parallel beam of radiation of a head 1 is referred to a slit lamp, which focuses radiation in a place of affecting on a biotissue.

The cross-section dimension of range of affecting is defined by diameter of spot focused (coagulating) laser radiation. The arrangement of the cord terminal 4 with the arrangement of the catch and the arrangement of a slit lamp allows to control the dimension of focused spot and by that the dimension of a coagulation area.

The emission which has got out the output end of a fibre cord terminal 4, penetrates into biotissues, for example, in a cornea of an eye. In process of diffusion of radiation its intensity Iₑₘᵢₜ decreases on exponential law

Iₑₘᵢₜ=I₀e^{αx},

wherein I₀ is a radiation intensity on a outlet of a cord terminal 4, α is an absorption coefficient, x is a distance. The reciprocal of absorption coefficient L=α⁻¹ defines a penetration depth of radiation in a biotissue. Experience shows, that the penetration depth of radiation L in a cornea can vary from 170 up to 600 microns at change of a wavelength within the limits of 1.34-1.44 microns.

In the arrangement of laser ophthalmologic thermocoagulator it is offered to use the local refrigerating of biotissues in a place exposed to coagulating radiation (FIG. 1). It is attained by use of a transparent heat-conducting sheet 6, the block of refrigerating of a sheet 7 and the block of an exposure of refrigerating 8. The block of an exposure of refrigerating 8 electrically is connected with the power package 2 and applies a signal of switching of refrigerating on it.

Local refrigerating of biotissues is carried out as follows. Preliminarily a sheet 6 is being cooled by means of the block 7 containing, for example, element Peltier. The sheet 6 is in thermal contact with element Peltier and is cooled till, for example, up to temperature T=10° C., much lower, than coagulation point of biotissues T=35-41° C. At the moment of a contact of a sheet 6 to biotissues, the torque motor of contact 14 operates. The signal from this sensing transducer is transmitted with some time delay to the power package 2, which operates from this signal and switches on the laser 1. Radiation from an outlet of the cord terminal 4 or a slit lamp passes through a transparent sheet 6 and processing of heating of tissues begins. Running time of a laser 1 and the related process of coagulation of biotissues is controlled by means of the electrical power unit 2.

On FIG. 5 the temperature distribution in a cross-section of a cornea of the eye, which is being contact to a sheet 6 is presented. First the sheet 6 of temperature Tₛₕₑₑₜ=1-15° C. contacts a cornea of temperature near to temperature of a human body. After a while the surface of a cornea will cool approximately up to Tₛₕₑₑₜ. Meanwhile the temperature of an intrinsic surface of a cornea will not essentially vary. In that moment (a priori chosen experimentally) by means of the block 8 a laser radiation is switched on, and the cornea starts to be heated non-uniformly. The outer surface of a cornea will more strongly be heated. Owing to precooling the surface temperature of a cornea will not achieve coagulation point T_{coag} (FIG. 5, the curve 20). Inside of a cornea of a biotissue has not time to cool so considerably as on a surface, therefore it will be heated to the temperatures exceeding coagulation point. The range, where there was coagulation, matches the cross-hatched part of the curve 20 on FIG. 5. Near to an intrinsic surface of a cornea a heating by a laser radiation is low therefore coagulation is absent.

Advantage of use of preliminary refrigerating contact of a sheet 6 with a surface of a cornea is absence of coagulation of a surface of a cornea and, accordingly, a prevention of undesirable, in some cases, a damage.

Curves 21-24 on FIG. 5 illustrate change of temperature distribution in a cross-section of a cornea in process of augmentation of absorption coefficient α. At small α (the curve 21) temperature distribution is more uniform, but the appreciable part of a laser radiation passes through a cornea, and there is a danger of a damage of the tissues laying behind a cornea. In case of strong absorption (the curve 24), radiation is absorbed non-uniform, however a positive effect is that it practically does not pass through a cornea. By varying a radiation wavelength it is possible to change essentially a profile of the coagulated volume of biotissues and by that (depending on a task in view) to optimise the conditions of action on the biotissues.

At the additional use of one or several diode lasers with wavelengths of emitting in ranges of 0.47-0.49 microns, 0.62-0.65 microns, 0.8-1.06 microns, 1.25-1.29 microns besides heating biotissues are simultaneously provided a medical therapeutic effect. If necessary the declared arrangement can be used only for therapeutic effect.

The maximum of efficiency of a radiation effect on erythrocytes of the blood resulting in their maximal elasticity and accordingly to appreciable enriching the blood microcirculation in capillaries matches to a range of wavelengths 1.25-1.29 microns. It results in the decreasing of periods of healing of the various diseases, including an adhesion after laser coagulation.

In the declared arrangement the use of the cord terminal 4 shown on FIG. 6 is effective. The end of a cord terminal 4 of an optical fibre 25 of a diameter within the limits of 0.05-1.00 mm bulges on some controlled distance L. This end 25 is executed without protective and reflecting coatings and on face and lateral surfaces contains embedment of light-absorbing corpuscles, for example, a graphitic powder. Alternative is also a drawing of any heat-resistant light-absorbing coating. In particular, the surface of the end of an optical fibre 25 can be metallised, for example, with aluminium. Under action of a laser radiation the embedment of light-absorbing corpuscles or a light-absorbing coating are heated. The end of an optical fibre 25 is heated as result. Such the cord terminal 4 (FIG. 6) can be used simultaneously as for coagulation and ablation, and cutting of biotissues. It can be effective also in thermokeratoplasty. Unlike a cord terminal with the metal needle heated by a high-frequency electromagnetic field, the power density supplied to the end of an optical fibre 25, considerably higher and accordingly higher an efficiency of action of the optically heated cord terminal. The cord terminal 4 (FIG. 6) is more compact. Besides it is very effective at cutting of various soft biotissues. Varying length L (FIG. 6), it is possible to change a cutting depth of soft tissues effectively. It is especially important at executing of operations on correction of vision by thermokeratoplasty.

At use of declared inventions unlike the proximate analogue a full recovery at all patients at a dartrous keratitis occurs much more fast (within 2 weeks). Moreover, it has appeared, that for full recovery the laser thermocoagulation needs to be carried out once.

For testing of the offered method the following experimental researches have been carried out. On the faculty of ophthalmic diseases of Russian Friendship University named after Patrice Lumumba are carried out the researches of studying of medical action of the coagulating laser radiation with a wavelength is within the absorption range of a cornea of an eye at dartrous a keratitis. The testing was carried out on 10 patients in the age of from 18 until 60 years which have entered in individual way in the advisory-diagnostic centre of the Medical-sanitary unit where they were high-grade diagnosed and were under treatment in the outpatient department. At patients it was observed the superficial dartrous lesion of a cornea of the eye, badly responsive to conservative treatment in outpatient conditions. The one-stage laser coagulation of dartrous elements (blisters) of surface layers of a cornea has been led to the patients. As a source of laser coagulating radiation the arrangement shown on FIG. 2 has been used. In a laser head 1 the two laser diodes 15 of the POLAROID company (USA) of the power 500 mW each were used. One of lasers 15 had a wavelength of radiation 1.44 microns, and another—1.40 microns. The greatest radiated power of each of lasers 15 on an output of a cord terminal 4 achieved 400 mW at 400 microns diameter of a core of an optical fibre used for delivery of radiation. The end of a fibre 25 placed on distance 2-6 mm from a cornea. Exposure time of one cycle of action of a laser radiation on a cornea was 0.5-1.5 sec. At typical diameter of laser radiation spot on a cornea of an eye from 1 up to 4 mm, the process of laser action totally on the dartrous disturbed surface consisted from 10-30 exposures. All process of laser coagulation of a surface of a cornea took 0.5-5 minutes. The best result was attained at use of the laser 15 with wavelength 1.40 microns. At use of this wavelength more effective coagulation of tissues on big depth was carried out. After laser action to a cornea the standard plan of treatment was used (i.e., antibiotics, etc.). During observation (two weeks) the full epithelium of surface layers of a cornea, with objective enriching acuity of vision, and almost full absence of clinical effect of a dartrous keratitis (the residual hyperemia of a conjunctiva) was observed.

Patients are exposed the routine inspections in Advisory-Diagnostic Centre, the relapses of disease has not been taped. The obtained results in the early term testify about good prognosis, safety, stability of the proposed method of treatment in a combination with the complex, conservative therapy, and also about an opportunity of treatment of patients with dartrous lesion of eyes in conditions of a health centre.

What is claimed is:

1. The method of curing including the action on a biotissue by a laser radiation characterized in that a biotissue is attacked by a laser radiation of wavelength of ranges at least in one from the group 0.96-1.06 microns, 1.34-1.70 microns, 1.8-2.2 microns, 2.4-3.1 microns.

2. A method according to claim 1, characterized in that the biotissues are attacked by a continuous laser radiation.

3. A method according to claim 1, characterized in that the biotissues are attacked by a pulse laser radiation.

4. A method according to claim 1, characterized in that the biotissues are attack by a laser radiation which wavelength is varied during the exposure.

5. A method according to claim 4, characterized in that the wavelength of laser radiation is varied continuously

6. A method according to claim 4, characterized in that the wavelength of laser radiation is varied discretely

7. A method according to claim 1, characterized in that the biotissues are attacked by an additional laser radiation of wavelength of ranges at least in one from group 0.47-0.49 microns, 0.62-0.65 microns, 0.8-0.95 microns, 1.25-1.29 microns.

8. The arrangement for the curing containing a laser head with the power supply block, the block of delivery and focusing of the radiation, containing a fibre-optic cable on which output the cord terminal is mounted, characterized in that the laser head is executed with an opportunity of emission on various wavelengths within the limits of, at least, one absorption band of biotissues, and the block of delivery and focusing of radiation additionally contains a transparent heat-conducting sheet, the block of refrigerating of a sheet and the block of an exposure of refrigerating.

9. The device according to claim 8, characterized in that the laser emitter is executed with an opportunity of alteration of wavelength within the limits of, at least, one line of absorption of biotissues.

10. The arrangement according to claim 8, characterized in that the laser emitter contains not less than two lasers emitting on various wavelengths.

11. The arrangement according to claim 10, characterized in that it contains the diode laser.

12. The arrangement according to claim 10, characterized in that it contains the fibre laser.

13. The arrangement according to claim 10, characterized in that it contains the solid-state laser.

14. The arrangement according to claim 13, characterized in that it contains the laser which host of an active element is executed from a single crystal from the group YAlO₃, Y₃Al₅O₁₂, YVO₄, GdVO₄, LiYF₄.

15. The arrangement according to claim 14, characterized in that the optically active element of the laser is doped at least by one ion from group Nd³⁺, Pr³⁺, Ho³⁺, Er³⁺, Tm³⁺, Yb³⁺, Ti³⁺, Cr³⁺, Cr⁴⁺.

16. The arrangement according to claim 8, characterized in that the laser emitter contains the block of lamp and/or diode pumping.

17. The arrangement according to claim 8, characterized in that it additionally contains the beam convergence block of radiation on various wavelengths in one beam.

18. The arrangement according to claim 8, characterized in that it contains the source of an additional laser radiation executed in the form of the semiconductor laser.

19. The arrangement according to claim 5, characterized in that the block of focusing and delivery of radiation is electrically connected with the power supply block.

20. The arrangement according to claim 5, characterized in that the cord terminal contains the case and it is mounted in such a manner that the optical fibre bulges out of the case.

21. The arrangement according to claim 20, characterized in that the optical fibre bulges out of the case on the controlled distance.

22. The arrangement according to claim 20, characterized in that the face and lateral cylindrical surfaces of the optical fibre bulging out of the case, absorb not less than 10% of power of radiation supplied.

23. The arrangement according to claim 8, characterized in that the transparent heat-conducting sheet is mounted behind the cord terminal and it is connected with the block of refrigerating of a sheet.

24. The arrangement according to claim 8, characterized in that the block of refrigerating is electrically connected with the block of an exposure of refrigerating.

25. The arrangement according to claim 8, characterized in that it additionally contains a thermal transducer mounted on a heat-conducting sheet.
